(19) United States
(12) Patent Application Publication
(10) Pub. No.: US 2005/0070796 A1
(43) Pub. Date: Mar. 31, 2005

(54) Ultrasonic diagnosing apparatus

(75) Inventor: Kazuhiro Tsujita, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/947,301

(22) Filed: Sep. 23, 2004

(30) Foreign Application Priority Data
Sep. 30, 2003 (JP) 2003-340737

(51) Int. Cl. A61B 8/00

(52) U.S. Cl. 600/437

(57) ABSTRACT

An ultrasonic diagnosing apparatus capable of obtaining more image information on living tissues by utilizing attenuation information of ultrasonic waves at plural frequencies as information on tissues within an object to be inspected. The ultrasonic diagnosing apparatus includes: a separating unit for separating a frequency component of a signal obtained by transmitting ultrasonic waves to the object and receiving the ultrasonic waves reflected from the object or transmitted through the object, into frequency components at different frequencies or in different frequency bands to obtain plural frequency components; a computing unit for obtaining relative relationships between intensity of the plural frequency components obtained by the separating unit at different points of time to obtain changes in the relative relationships between the intensity; and an image data generating unit for generating image data on the object based on the changes in the relative relationships between the intensity obtained by the computing unit.

### BACKGROUND OF THE INVENTION

1. Field of the Invention

The present invention relates to an ultrasonic diagnosing apparatus for performing imaging of organs, bones, etc. within a living body by transmitting and receiving ultrasonic waves so as to generate ultrasonic images used for diagnosis.

2. Description of a Related Art

In an ultrasonic diagnosing apparatus used for medical application, normally, an ultrasonic probe including plural ultrasonic transducers having the transmitting and receiving functions of ultrasonic waves is used. Using such ultrasonic probe, an object to be inspected is scanned by an ultrasonic beam formed by synthesizing the ultrasonic waves transmitted from the plural ultrasonic transducers and the ultrasonic echoes reflected inside the object are received, and thereby, image information on the object is obtained based on the intensity of the ultrasonic echoes. Furthermore, two-dimensional or three-dimensional images on the object are reproduced based on the image information.

By the way, a human body includes various tissues such as soft tissues like muscles and hard tissues like bones. In the ultrasonic imaging, it is conceivable that plural frequency components included in the ultrasonic echoes are utilized as information for distinguishing these tissues.

As a related technology, JP-A-2-206446 discloses an ultrasonic diagnosing apparatus capable of reducing speckle components generated with a result that a large number of weak echoes are added and interfere, and thereby, obtaining ultrasonic images with high image quality. In this ultrasonic diagnosing apparatus, plural transmission signals corresponding to different transmission frequencies are transmitted with respect to each ultrasonic raster, and each reception signal reflected from the object is filtered at a frequency band corresponding thereto. Thereby, interferences differ between ultrasonic rasters, so that there is no correlation between ultrasonic rasters. As a result, there is no correlation between ultrasonic rasters with respect to speckles, and thereby, speckles can be reduced. However, there has been a problem that the frame rate is reduced by transmitting plural transmission signals corresponding to different transmission frequencies. Further, there has been no suggestion on utilization of plural frequency components in each ultrasonic raster.

Further, JP-A-2001-170049 discloses an ultrasonic diagnosing apparatus for reducing deterioration of spatial resolution in a distance direction in the case where speckle reduction is performed according to the frequency compound system. In this ultrasonic diagnosing apparatus, from reception signals, plural narrow-band signal components are extracted by narrow-band pass filters different from each other and a broad-band signal component is extracted by a broad-band pass filter, and those signal components are weighted and added. Since a broad band containing plural narrowbands is set other than those narrowbands, the reduction of spatial resolution in the distance direction can be accommodated. However,there is no suggestion on utilization of attenuation information of ultrasonic waves at plural frequencies as information on tissues within the object.

### SUMMARY OF THE INVENTION

An object of the present invention is to provide an ultrasonic diagnosing apparatus capable of obtaining more imaging information on living tissues by utilizing attenuation information of ultrasonic waves at plural frequencies as information on tissues within the object.

In order to solve the above-described problems, an ultrasonic diagnosing apparatus according to the present invention includes: a separating unit for separating a frequency component of a signal obtained by transmitting ultrasonic waves to an object to be inspected and receiving the ultrasonic waves reflected from the object or transmitted through the object, into frequency components at different frequencies or in different frequency bands so as to obtain a plurality of frequency components; a computing unit for obtaining relative relationships between intensity of the plurality of frequency components obtained by the separating unit, at a plurality of different points of time so as to obtain changes in the relative relationships between the intensity; and an image data generating unit for generating image data on the object based on the changes in the relative relationships between the intensity obtained by the computing unit.

According to the present invention, the frequency components of the signal obtained by transmitting and receiving the ultrasonic waves are separated into the frequency components at different frequencies or in different frequency bands, the relative relationships between the intensity of the obtained plural frequency components are obtained at different plural points of time, image data is generated based on the changes in the obtained relative relationships between the intensity, and thereby, more imaging information can be obtained with respect to living tissues by utilizing the attenuation information of the ultrasonic waves at plural frequencies as information on tissues within the object.

### BRIEF DESCRIPTION OF THE DRAWINGS

FIG. 1 is a block diagram showing the constitution of an ultrasonic diagnosing apparatus according to the first embodiment of the present invention;

FIG. 2 shows the frequency characteristics of sound ray signals with respect to plural different tissues measured at plural different times, which are obtained by transmitting and receiving burst signals of ultrasonic waves;

FIG. 3 shows the waveforms of two frequency components included in the sound ray signals obtained by transmitting and receiving the burst signals of ultrasonic waves;

FIG. 4A schematically shows an example of a B-mode image displayed in the ultrasonic diagnosing apparatus according to the first embodiment;

FIG. 4B schematically shows an example of a frequency image;

FIG. 4C schematically shows an example of a synthesized image of the B-mode image and the frequency image;

FIG. 5 is a block diagram showing the constitution of an ultrasonic diagnosing apparatus according to the second embodiment of the present invention;

FIG. 6 shows the frequency characteristics of the reception signals with respect to the object measured at different plural times, which are obtained by transmitting and receiving burst signals of ultrasonic waves; and

FIG. 7 shows a transmission-type ultrasonic probe performing rotationally scanning the object.

### DESCRIPTION OF THE PREFERRED EMBODIMENTS

Now, the best mode for carrying out the present invention will be described in detail with reference to the drawings. Incidentally, identical reference numerals are assigned to the same constituents, which shall be omitted from description.

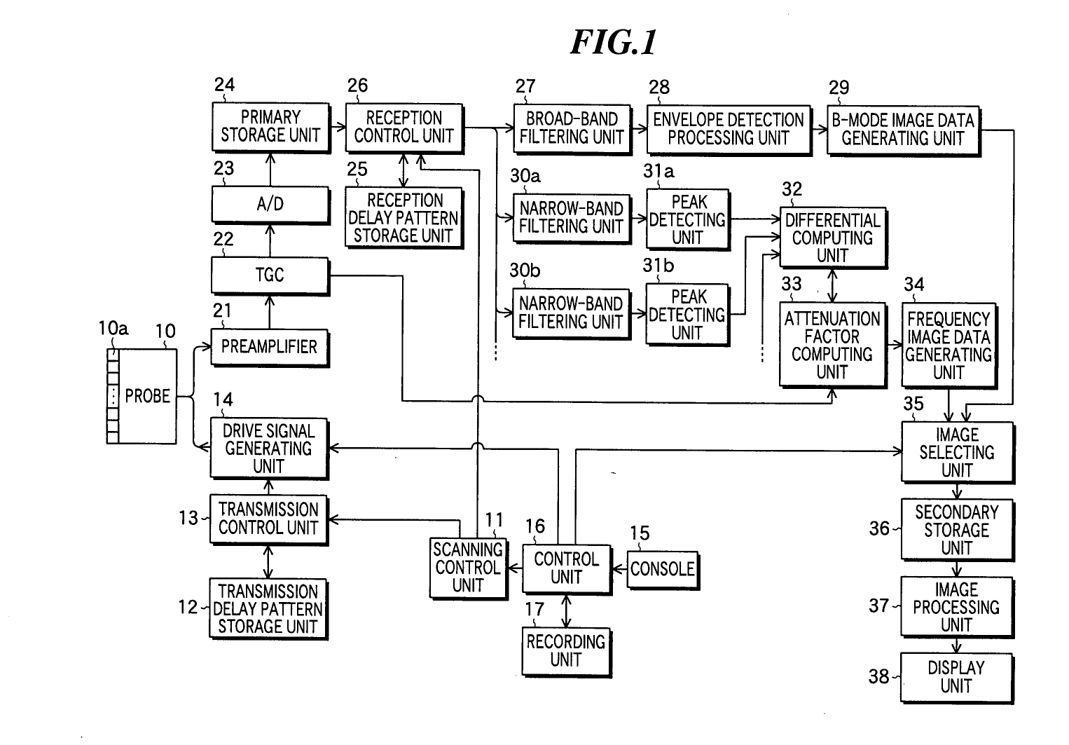

FIG. 1 is a block diagram showing the constitution of an ultrasonic diagnosing apparatus according to the first embodiment of the present invention. The ultrasonic diagnosing apparatus according to the embodiment includes an ultrasonic probe 10, a scanning control unit 11, a transmission delay pattern storage unit 12, a transmission control unit 13 and a drive signal generating unit 14.

The ultrasonic probe 10 used by being abutted on an object to be inspected includes plural ultrasonic transducers 10a arranged in a one-dimensional or two-dimensional manner that form a transducer array. These ultrasonic transducers 10a transmit ultrasonic beams based on applied drive signals, and receive ultrasonic echoes from inside the object and output detection signals.

Each ultrasonic transducer 10a is constituted by a vibrator in which electrodes are formed on both ends of a material having a piezoelectric property (piezoelectric element) such as a piezoelectric ceramic represented by PZT (Pb (lead) zirconate titanate), a polymeric piezoelectric element represented by PVDF (Polyvinylidene difluoride), or the like. When a voltage is applied to the electrodes of the vibrator by transmitting pulse electric signals or continuous wave electric signals, the piezoelectric element expands and contracts. By the expansion and contraction, pulse ultrasonic waves or continuous wave ultrasonic waves are generated from the respective vibrators, and an ultrasonic beam is formed by synthesizing these ultrasonic waves. Further, the respective vibrators expand and contract by receiving the ultrasonic echoes from inside the object and generate electric signals. These electric signals are outputted as the detection signals of the ultrasonic echoes.

Alternatively, as the ultrasonic transducers 10a, plural kinds of elements of different conversion types may be used. For example, the above-described vibrators are used as elements for transmitting the ultrasonic waves and photo-detection type ultrasonic transducers are used as elements for receiving the ultrasonic waves. The photo-detection type ultrasonic transducer is for detecting the ultrasonic waves by converting ultrasonic signals into optical signals, and constituted by a Fabry-Perot resonator or fiber Bragg grating, for example.

The scanning control unit 11 sets the transmission direction of the ultrasonic beams and the reception direction of the ultrasonic echoes sequentially. The transmission delay pattern storage unit 12 has stored plural transmission delay patterns used when the ultrasonic beams are formed. The transmission control unit 13 selects a predetermined pattern of the plural delay patterns that have been stored in the transmission delay pattern storage unit 12, in response to the transmission direction set in the scanning control unit 11, and sets delay times of the drive signals, which are provided to the plural ultrasonic transducers 10a, based on the pattern.

The drive signal generating unit 14 is constituted by a signal generating circuit for generating signals having plural frequency components such as burst signals or frequency multiple signals, and plural drive circuits for providing desired delays to the signals generated by the signal generating circuit and generating plural drive signals to be supplied to the plural ultrasonic transducers 10a, respectively. These drive circuits delay the signals generated by the signal generating circuit, based on the delay times set in the transmission control unit 13.

Further, the ultrasonic diagnosing apparatus according to the embodiment includes a console 15, a control unit 16 having a CPU, and a recording unit 17 such as a hard disk. The control unit 16 controls the scanning control unit 11, the drive signal generating unit 14 and an image selecting unit 35 based on the operation by the operator using the console 15. In the recording unit 17, programs for allowing the CPU, which forms the control unit 16, to execute various kinds of operation, and the frequency characteristics in the transmission and reception of the ultrasonic transducers 10a are recorded.

Furthermore, the ultrasonic diagnosing apparatus according to the embodiment includes a preamplifier 21, a TGC (Time gain compensation) amplifier 22, an A/D (analog/digital) converter 23, a primary storage unit 24, a reception delay pattern storage unit 25, a reception control unit 26, a broad-band filtering unit 27, an envelope detection processing unit 28, a B-mode image data generating unit 29, narrow-band filtering units 30a, 30b, . . . , peak detecting units 31a, 31b, . . . , a differential computing unit 32, an attenuation factor computing unit 33, a frequency image data generating unit 34, the image selecting unit 35, a secondary storage unit 36, an image processing unit 37, and a display unit 38.

The detection signals of the ultrasonic echoes outputted from the respective plural ultrasonic transducers 10a are amplified by the preamplifier 21, and corrected the attenuation of the ultrasonic waves in response to the distance that the ultrasonic waves reach within the object, by the TGC amplifier 22.

The analog detection signals outputted from the TGC amplifier 22 are converted into digital detection signals by the A/D converter 23. As a sampling frequency of the A/D converter 23, at least about a tenfold frequency of the frequency of the ultrasonic wave is required, and a 16-fold or more frequency of the frequency of the ultrasonic wave is desirable. Further, as the resolving power of the A/D converter 23, a resolving power of ten or more bits is desirable. The primary storage unit 24 stores the digital detection signals outputted from the A/D converter 23 with respect to each ultrasonic transducer 10a in chronological order.

The reception delay pattern storage unit 25 has stored plural reception delay patterns used when the reception focusing processing is performed on the plural detection signals outputted from the plural ultrasonic transducers 10a. The reception control unit 26 performs the reception focusing processing by selecting a predetermined pattern of the plural delay patterns, which have been stored in the reception delay pattern storage unit 25, in response to the reception direction set in the scanning control unit 11, and providing the delay times to the plural detection signals based on the pattern and adding the signals. By the reception focusing processing, sound ray data representing sound ray signals in which the focus of the ultrasonic echo is narrowed is formed. By the way, the reception focusing processing may be performed before the A/D conversion of the detection signals by the A/D converter 23 or the correction of the detection signals by the TGC amplifier 22.

The broad-band filtering unit 27 performs the broad-band band-pass filter processing on the sound ray data outputted from the reception control unit 26. The envelope detection processing unit 28 performs the envelope detection processing on the sound ray data subjected to the broad-band filter processing, and obtains envelope data representing the envelopes of the sound ray signals. The B-mode image data generating unit 29 generates B-mode image data based on the envelope data of the sound ray signals. By the way, the broad-band filtering unit 27 may be omitted, and data formed by synthesizing plural frequency components obtained by the narrow-band band-pass filter processing by the narrow-band filtering units 30a, 30b, . . . maybe generated so as to generate the B-mode image data based on the data.

The narrow-band filtering units 30a, 30b, . . . obtain plural frequency components by performing the narrow-band band-pass filter processing, which are different in passing bands from each other, on the sound ray data outputted from the reception control unit 26 so as to separate the frequency components of the sound ray signals into frequency components at different frequencies or in different frequency bands. The peak detecting units 31a, 31b, . . . detect the peaks of the plural frequency components outputted from the respective narrow-band filtering units 30a, 30b, . . . , and obtain the peak values of the plural frequency components at plural points of time.

The differential computing unit 32 computes the differences with respect to the peak values of the plural frequency components at each point of time, and thereby, obtains the differences between these peak values. Furthermore, the attenuation factor computing unit 33 computes the amounts of the variation of the differences between these peak values at plural points of time, and thereby, obtains attenuation information of the ultrasonic waves between plural frequencies. Thus, the attenuation information of the ultrasonic waves between plural frequencies is obtained based on the changes in the relative relationships between the intensity of the plural frequency components of the sound ray signals included in the sound ray data. The attenuation information of the ultrasonic waves is utilized as information on tissues within the object.

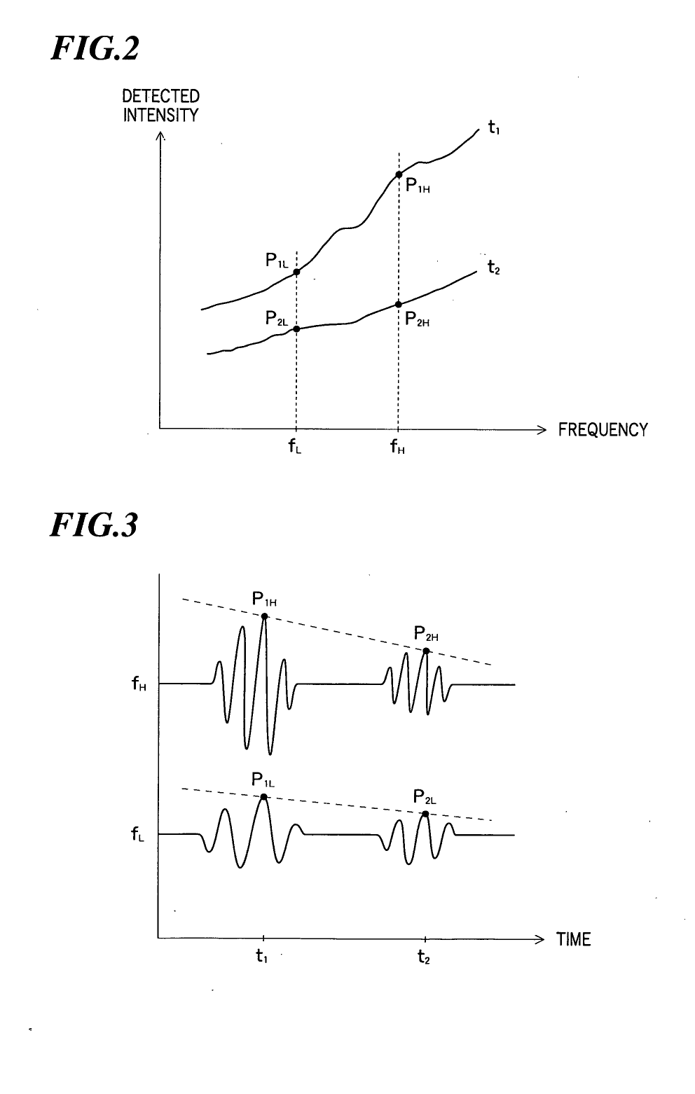

FIG. 2 shows the frequency characteristics of the sound ray signals with respect to plural different tissues measured at plural different times, which are obtained by transmitting and receiving the burst signals of ultrasonic waves, and FIG. 3 shows the waveforms of two frequency components included in the sound ray signals. As shown in FIG. 2, the sound ray signals obtained by transmitting and receiving the burst signals of ultrasonic waves have the broad-band frequency components. Among them, attention is focused on low frequency components at frequency f_{L} and high frequency components at frequency f_{H}.

As shown in FIG. 3, strong ultrasonic echoes are observed at time point t₁ and time point t₂, and these parts show that the ultrasonic waves are reflected in parts where the reflectance is large like at the interfaces between soft tissues (muscles or the like) and hard tissues (bones or the like) of the object. The attenuation characteristics of the ultrasonic waves in the tissue sandwiched between two interfaces within the object can be obtained by separating the frequency components of the sound ray signals obtained by transmitting and receiving the burst signals of ultrasonic waves into frequency components at plural frequencies (low frequency components at frequency f_{L} and high frequency components at frequency f_{H}), and measuring the intensity of the separated plural frequency components.

In FIGS. 2 and 3, the intensity of the low and high frequency components at time point t₁ are given as P_{1L} and P_{1H}, respectively, and the intensity of the low and high frequency components at time point t₂ are given as P_{2L} and P_{2H}, respectively. In the ultrasonic diagnosing apparatus according to the embodiment, the intensity of the separated plural frequency components are obtained as peak values, however, the intensity of the separated plural frequency components may be obtained as PV values (peak-to-valley values), effective values, integral values, or the like.

In the example shown in FIG. 3, with respect to the low and high frequency components, intensity P_{2L} and P_{2H} at time point t₂ are smaller than intensity P_{1L} and P_{1H} at time point t₁. Although this does not directly correspond to the attenuation of the ultrasonic waves, the frequency characteristics in the attenuation of the ultrasonic waves can be obtained by calculating the changes in the intensity differences of the plural frequency components.

If the gains until the intensity in the reflection points of the ultrasonic waves is converted into the intensity of the sound ray signals are given as G₁ with respect to the sound ray signals measured at time point t₁, and given as G₂ with respect to the sound ray signals measured at time point t₂, respectively, the frequency characteristics in the attenuation of the ultrasonic waves per unit time in time Δt from time point t₁ to time point t₂are expressed by following equation (1).

$$\{ ( {P}_{2 H} / {G}_{2} - {P}_{1 H} / {G}_{1} ) - ( {P}_{2 L} / {G}_{2} - {P}_{1 L} / {G}_{1} ) \} / Δ t = \{ ( {P}_{2 H} - {P}_{2 L} ) / {G}_{2} - ( {P}_{1 H} - {P}_{1 L} ) / {G}_{1} \} / Δ t \qquad ( 1 )$$

Here, if the gains until the intensity in the reflection points of the ultrasonic waves is converted into the intensity of the sound ray signals are constant, following equation (2) can be used instead of equation (1).
{(P_{2H}−P_{2L})−(P_{1H}−P_{1L})}/Δt (2)
Furthermore, if P_{1H}=P_{1L}, following equation (3) can be used instead of equation (2). In this case, intensity difference (P_{2H}−P_{2L}) between the low frequency components and the high frequency component at time point t₂ represents the frequency characteristics in the attenuation of the ultrasonic waves.
(P_{2H}−P_{2L})/Δt (3)

Although the example in which the intensity differences of the plural frequency components are obtained as the relative relationships between the intensity of the plural frequency components of the sound ray signals has been described as above, the ratios between the intensity of the plural frequency components may be obtained. Since it is conceivable that the reflectance of the ultrasonic waves in tissues within the object do not very much depend on frequencies, if the attenuation characteristics are calculated by equation (1) or the like, there is an advantage that the attenuation characteristics can hardly be affected by the ultrasonic wave reflectance that vary depending on the differences between adjacent tissues within the object.

Further, as shown in equation (1), in the case where correction is performed on gains G₁ and G₂, the values corresponding to gains G₁ and G₂ can be obtained by utilizing control signals used for performing the correction of the attenuation in the TGC amplifier S22 shown in FIG. 1. Furthermore, if the frequency characteristics in the transmission and reception of the ultrasonic transducers 10a have been recorded in the recording unit 17 and the intensity of the plural frequency components of the sound ray signals are corrected in response to the frequency characteristics of the ultrasonic transducers 10a, more accurate attenuation characteristics can be calculated.

Thus, the differential computing unit 32 and the attenuation factor computing unit 33 can obtain information on tissues within the object such as differences between soft tissue and hard tissues and differences between tissues like between tendons and muscles within the soft tissue, based on plural frequency components having different attenuation characteristics of ultrasonic waves in tissues to be subjected to imaging. Based on this information, the frequency image data generating unit 34 generates frequency image data (spectrum image data).

The image selecting unit 35 synthesizes the B-mode image data generated by the B-mode image data generating unit 29 and the frequency image data generated by the frequency image data generating unit 34 or selects one of these data, and output the data. The secondary storage unit 36 stores the image data outputted from the image selecting unit 35. The image processing unit 37 performs various kinds of image processing on the image data stored in the secondary storage unit 36. The display unit 38 includes a display device such as a CRT or an LCD, and displays ultrasonic images based on the image data subjected to the image processing by the image processing unit 37.

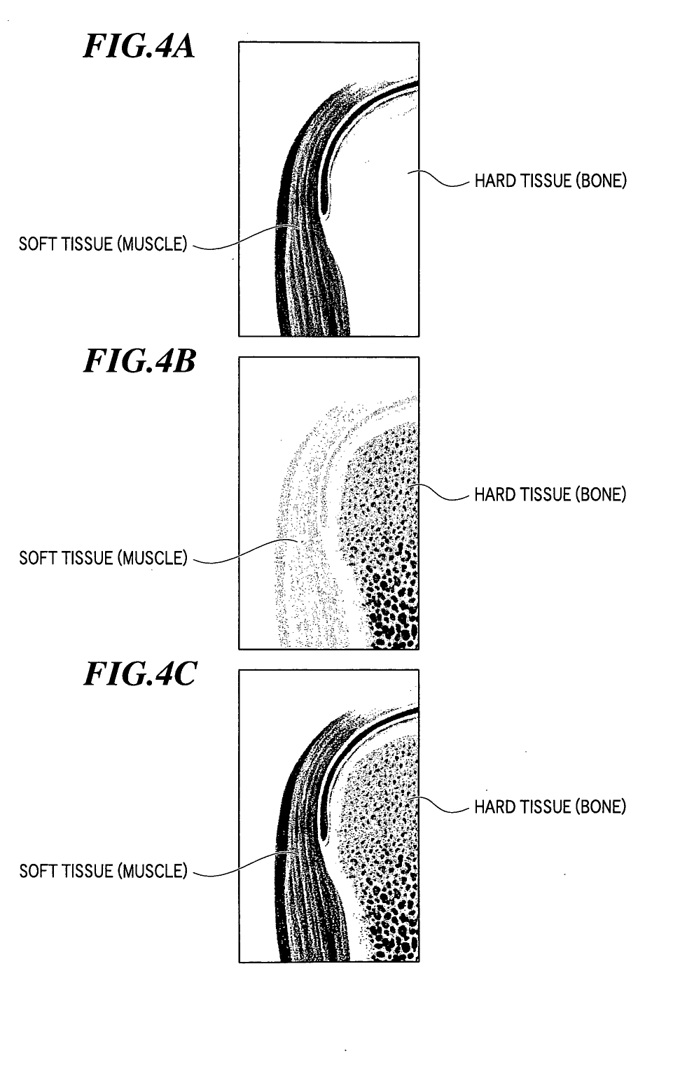

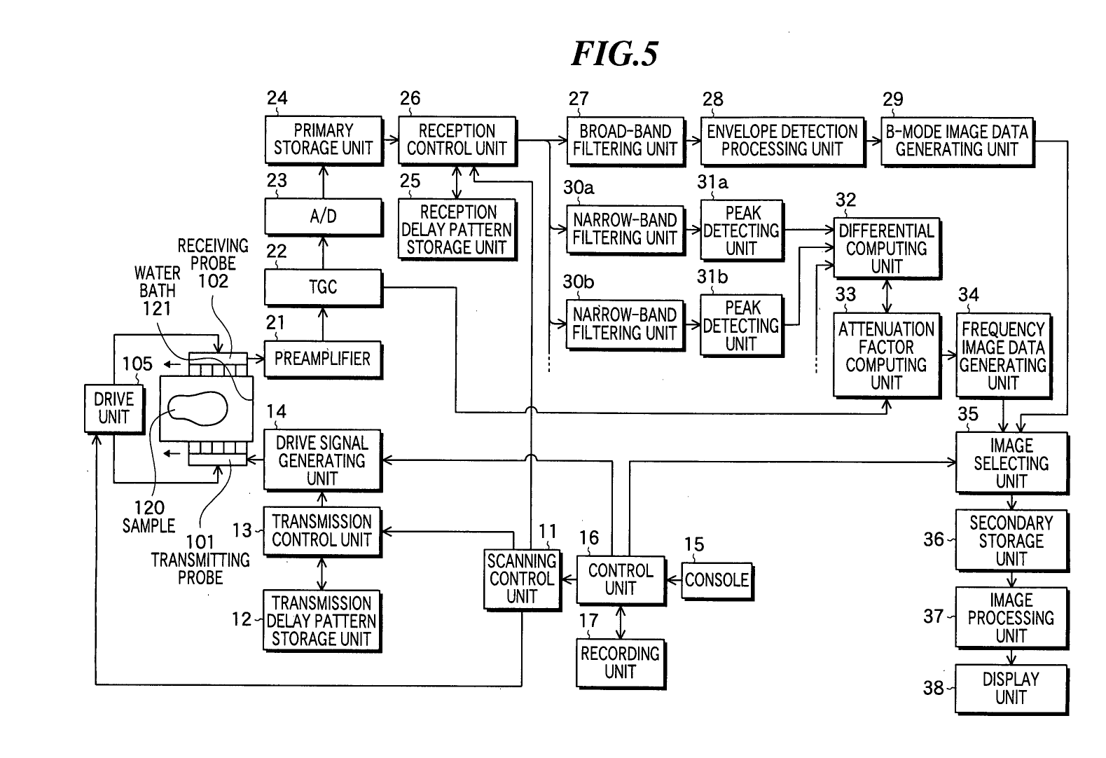

In FIGS. 4A to 4C, examples of the ultrasonic images displayed in the ultrasonic diagnosing apparatus according to the embodiment are schematically shown. FIG. 4A shows a B-mode image. The ultrasonic image in which the interior of the hard tissue (bone) is almost unclear, but the soft tissue (muscle) existing outside of the hard tissue (bone) is shown is generated. On the other hand, FIG. 4B shows a frequency image. The interior of the hard tissue (bone) can be emphatically displayed by extracting suitable frequency components. Further, the separation of the hard tissue (bone) from the soft tissue (muscle) is clearly shown, and imaging from the bone to the skin can be performed. FIG. 4C is a view displayed by synthesizing the B-mode image and the frequency image. For example, the image selecting unit 35 (see FIG. 1) may output luminance signals (or chromaticity signals) based on the B-mode image data generated by the B-mode image data generating unit 29, and chromaticity signals (or luminance signals) based on the frequency image data generated by the frequency image data generating unit 34. Further, the region of interest for displaying the attenuation factor information may be designated in the display screen.

In the ultrasonic diagnosing apparatus according to the above described embodiment, both sectional image information and attenuation factor information are simultaneously obtained by a single set of the transmission and reception of the ultrasonic waves by generating the drive signals having plural frequency components by the drive signal generating unit 14. However, the attenuation factor information of a signal frame may be obtained while obtaining sectional image information of plural frames by generating the drive signals having different frequency components with respect to each sound ray by the drive signal generating unit 14. Further, without performing the correction of the gains utilizing the control signals used in the TGC amplifier 22, only the relative values of the attenuation characteristics may be displayed or only the positive or negative of the attenuation characteristics may be determined.

Next, an ultrasonic diagnosing apparatus according to the second embodiment of the present invention will be described by referring to FIGS. 5 to 7.

The ultrasonic diagnosing apparatus according to the embodiment is different from the ultrasonic diagnosing apparatus according to the first embodiment shown in FIG. 1 in that it includes, instead of the ultrasonic probe 10, a transmission ultrasonic probe having an ultrasonic transmitting probe 101 for transmitting ultrasonic waves and an ultrasonic receiving probe 102 for receiving ultrasonic waves that perform translational or rotational scanning while keeping a state in which they are opposed with the object therebetween, and a drive unit 105 controlled by the scanning control unit 11 for translationally or rotationally driving the ultrasonic transmitting probe 101 and the ultrasonic receiving probe 102, and makes images by calculating the distribution of the frequency characteristics of the transmittance within a section of the object (difference value between the frequency characteristics of the transmittance)

In the ultrasonic diagnosing apparatus shown in FIG. 5, the ultrasonic receiving probe 102 receives ultrasonic waves, which are transmitted from the ultrasonic transmitting probe 101 and transmitted through a sample 120 within a water bath 121, while controlling the drive unit 105 by the scanning control unit 11 to translationally drive the ultrasonic transmitting probe 101 and the ultrasonic receiving probe 102.

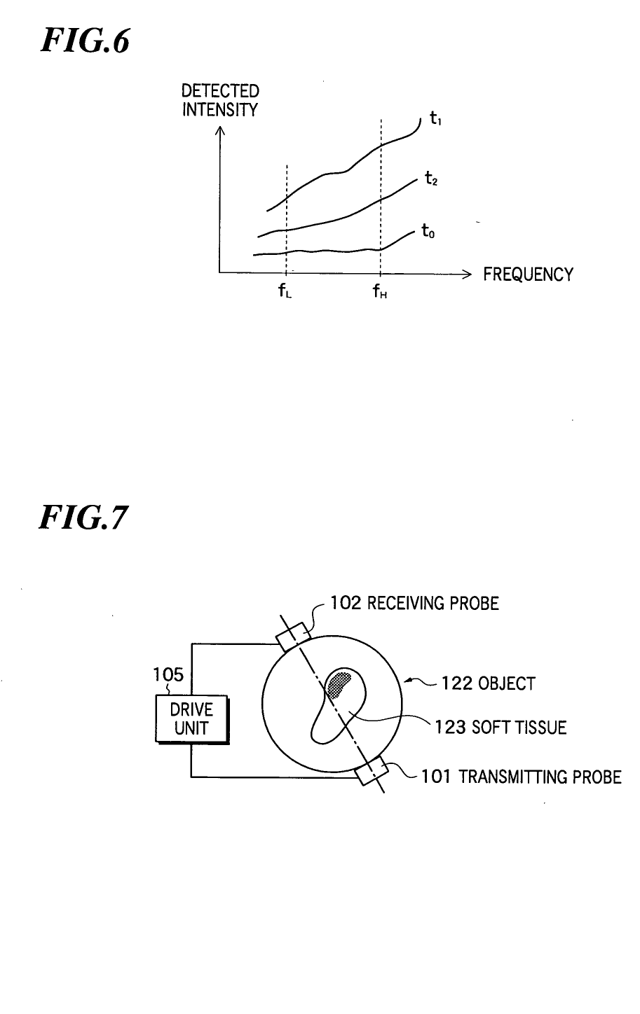

FIG. 6 shows the frequency characteristics of the reception signals with respect to the sample 120 measured at different plural times, which are obtained by transmitting and receiving the burst signals of ultrasonic waves. In the case where a transmission-type ultrasonic probe is used for measurement, the intensity of the frequency characteristics of the reception signals represent the frequency characteristics of the transmittance at times t₀, t₁, t₂, . . . . Further, in the case where a transmission-type ultrasonic probe is translationally scanned for measurement, times t₀, t₁, t₂, . . . represent the scanning positions of the transmission-type ultrasonic probe, and thereby, images showing the differences of the transmittances in the respective frequencies can be obtained by mapping the relative relationships between the intensity of the frequency characteristics of the transmittance while making them and the scanning positions correspond.

Thus obtained images showing the differences of the transmittances in the respective frequencies can represent the characteristics unique to samples even if the samples have different thickness, in comparison with the images showing the intensity of the ultrasonic waves simply transmitted through the sample 120. For example, if the position of the transmission-type ultrasonic probe corresponding to time to is set to a position where the ultrasonic waves are transmitted through the water only, but not transmitted through the sample 120, and the positions of the transmission-type ultrasonic probe corresponding to times t₁ and t₂ are set to positions where the ultrasonic waves are transmitted through the water and the sample 120, the frequency characteristics of the transmittance obtained at times t₁ and t₂ contain the frequency characteristics of the transmittance relative to a measurement system such as water and probes and the frequency characteristics of the transmittance relative to the sample 120.

In FIG. 5, the transmission-type ultrasonic probe performing the transitional scanning is used. However, as shown in FIG. 7, a transmission-type ultrasonic probe that rotationally scans an object to be inspected 122 while keeping a state in which the ultrasonic transmitting probe 101 and the ultrasonic receiving probe 102 are opposed with the object 122 therebetween may be used. In this case, similarly, the drive unit 105 is controlled by the scanning control unit 11 shown in FIG. 5 to drive the ultrasonic transmitting probe 101 and the ultrasonic receiving probe 102.

Here, when the position of the transmission-type ultrasonic probe corresponding to time t₀ is set to a part of a known muscle tissue, and the positions of the transmission-type ultrasonic probe corresponding to times t₁ and t₂ are set to parts including an unknown soft tissue 123, images showing the differences between the frequency characteristics of the transmittance obtained at time t₀ and the frequency characteristics of the transmittance obtained at times t₁ and t₂ are images showing the differences between the frequency characteristics of the transmittance in the soft tissue 123 relative to the muscle tissue. Thereby, the difference between the characteristics within the soft tissue becomes easier to be seen.

By the way, the distance between the ultrasonic transmitting probe 101 and the ultrasonic receiving probe 102 may be made adjustable so that they may be used by being abutted against the object 122.

The present invention can be utilized in an ultrasonic diagnosing apparatus for performing imaging of organs, bones, etc. within a living body by transmitting and receiving ultrasonic waves so as to generate ultrasonic images used for diagnosis.

1. An ultrasonic diagnosing apparatus comprising:
separating means for separating a frequency component of a signal obtained by transmitting ultrasonic waves to an object to be inspected and receiving the ultrasonic waves reflected from the object or transmitted through the object, into frequency components at different frequencies or in different frequency bands so as to obtain a plurality of frequency components;
computing means for obtaining relative relationships between intensity of the plurality of frequency components obtained by said separating means, at a plurality of different points of time so as to obtain changes in the relative relationships between the intensity; and
image data generating means for generating image data on the object based on the changes in the relative relationships between the intensity obtained by said computing means.

2. The ultrasonic diagnosing apparatus according to claim 1, said computing means includes a differential operation circuit for obtaining differences between the intensity of the plurality of frequency components obtained by said separating means.

3. The ultrasonic diagnosing apparatus according to claim 1, further comprising correcting means for correcting intensity of the signal obtained by transmitting the ultrasonic waves to the object and receiving the ultrasonic waves reflected from the object in response to a distance that the ultrasonic wave reaches within the object,
wherein said image data generating means corrects the changes in the relative relationships between the intensity based on information obtained in said correcting means.

4. The ultrasonic diagnosing apparatus according to claim 1, wherein said separating means performs a plurality of band-pass filter processing in which pass-bands are different from each other, on the signal obtained by transmitting the ultrasonic waves to the object and receiving the ultrasonic waves reflected from the object or transmitted through the object.

5. The ultrasonic diagnosing apparatus according to claim 4, wherein said image data generating means generates the image data based on the signal subjected to narrow-band band-pass filter processing by said separating means.

6. The ultrasonic diagnosing apparatus according to claim 1, further comprising:
other image data generating means for generating image data on the object based on intensity of the signal obtained by transmitting the ultrasonic waves to the object and receiving the ultrasonic waves reflected from the object or transmitted through the object; and
image selecting means for selecting at least one of the image data generated by said image data generating means and the image data generated by said other image data generating means.

7. The ultrasonic diagnosing apparatus according to claim 6, wherein said other image data generating means generates the image data based on the signal subjected to narrow-band band-pass filter processing and then envelope detection processing.

8. The ultrasonic diagnosing apparatus according to claim 6, wherein said image selecting means outputs a chromaticity signal based on the image data generated by said image data generating means and a luminance signal based on the image data generated by said other image data generating means.

9. The ultrasonic diagnosing apparatus according to claim 6, wherein said image selecting means outputs a luminance signal based on the image data generated by said image data generating means and a chromaticity signal based on the image data generated by said other image data generating means.

10. The ultrasonic diagnosing apparatus according to claim 1, wherein the relative relationships between the intensity of the plurality of frequency components are relative relationships between peak values, peak-to-valley values, effective values, or integral values of the plurality of frequency components.
